(19) United States
(12) Patent Application Publication
(10) Pub. No.: US 2013/0096568 A1
(43) Pub. Date: Apr. 18, 2013

(54) MODULAR TOOL APPARATUS AND METHOD

(71) Applicant: Jeff Justis, Germantown, TN (US)

(72) Inventor: Jeff Justis, Germantown, TN (US)

(73) Assignee: Warsaw Orthopedic, Inc., Warsaw, TN (US)

(21) Appl. No.: 13/276,082

(22) Filed: Oct. 18, 2011

(51) Int. Cl. A61B 17/56

(52) U.S. Cl. 606/104; 606/1

(57) ABSTRACT

A modular tool apparatus is provided having a handle defining a cannula extending at least partially therethrough and an elastic member disposed therein. The apparatus further provides a separate tool shaft configured to be releasably engaged with the handle by insertion into the cannula. The tool shaft comprises a plurality of teeth configured for ratcheting engagement with the elastic member when the tool shaft is inserted into the cannula. In some embodiments, the cannula extends completely through the handle from a proximal end to a distal end thereof to facilitate cleaning of the handle and separable tool shaft.

### BACKGROUND

The present invention relates in general to hand-held tool systems and in particular to a modular tool apparatus suitable for use as a more cleanable, steritizable, and reusable surgical instrument.

To stabilize a spinal system including a spinal column, the extent of displacement between adjacent vertebrae in the spinal column may be reduced, and/or each pair of adjacent vertebrae may be maintained in a desired spatial relationship using a surgical procedure.

In some cases, rods may be provided that are adapted to extend within the vicinity of the spinal system and connectors may be provided that connect one or more of the rods to one or more of the vertebrae in the spinal system and/or to iliac structures on the pelvis. The rods and connectors may assist in providing immobilization and/or stabilization to the spinal system, and/or may serve as an adjunct to fusion of one or more portions of the spinal system. An example of a system for reducing displacement of a vertebra, in which a rod is employed, is disclosed in U.S. Pat. No. 6,248,107 to Foley et al., the disclosure of which is incorporated by reference.

For spinal stabilization systems that include one or more rods connected to screws or other fasteners attaching the stabilization system to the spine, various tools having modular handles connectable to a variety of tool shafts may be desired and/or required in order to more easily engage set screws and bone screws, among other desires and/or requirements. Furthermore, tools being at least partially separable and easily cleanable and/or sterilizable may also be desired and/or required in order to more easily facilitate their use in a surgical setting requiring a sterile and/or semi-sterile working environment.

### SUMMARY

According to one exemplary embodiment, a modular tool apparatus extending along a longitudinal axis is provided, wherein the apparatus comprises a handle defining a cannula extending at least partially therethrough along the longitudinal axis. The handle also comprises an elastic member disposed therein. The modular tool apparatus also comprises a tool shaft configured to be releasably engaged with the handle by insertion into the cannula. The tool shaft comprises a plurality of teeth configured for ratcheting engagement with the elastic member when the tool shaft is inserted into the cannula, such that when the handle is rotated about the longitudinal axis in a first direction, the tool shaft rotates with the handle. Furthermore, when the handle is rotated about the longitudinal axis in a second direction, the tool shaft rotates relative to the handle. In various embodiments, the handle may comprise an inner rigid frame configured for retaining the elastic member and an outer handle suitable for gripping by a user.

According to another exemplary embodiment, the cannula defined in the handle extends completely therethrough from a proximal end to a distal end of the handle so as to facilitate thorough cleaning and/or sterilization of the various components of the modular tool apparatus to enable the apparatus to be used and re-used in surgical procedures within a sterile field.

In some embodiments, the elastic member disposed within the handle comprises a pawl and a spring configured to bias the pawl towards the plurality of teeth of the tool shaft when the tool shaft is inserted into the cannula. Furthermore, in some embodiments, the handle further comprises a retention member engaged therewith, wherein the retention member is configured for releasably engaging the toot shaft with the handle when the tool shaft is inserted into the cannula. In one exemplary embodiment, the retention member comprises: a tab extending through the handle, the tab defining a channel therein, the channel being at least partially co-axial with the cannula; and a biasing element configured for biasing the tab in a first position wherein the tab engages at least a portion of the tab with the tool shaft such that the tool shaft is releasably engaged with the handle within the cannula. In such embodiments, when the tab is pressed against the biasing element to a second position, the tool shaft is removable from the handle along the longitudinal axis.

According to various embodiments, the tool shaft may be modified to suit different toot configurations. For example, the tool shaft may comprise a cylinder having a first diameter, wherein the tool shaft comprises a distal end and a proximal end, wherein the proximal end has a second diameter larger than the first diameter such that the proximal end is releasably retained by the retention member when the tool shaft is engaged with the handle. Furthermore, the tool shaft may be tapered distally to the plurality of teeth disposed thereon. In some embodiments, the tool shaft may have a distal end comprising a fastener driving element that may include, but is not limited to: a multilobular male driver; a multilobular female driver; a flat-head male driver; a Philips head male driver a polygonal male driver; and a polygonal female driver.

In some further embodiments, the apparatus may further comprise a T-handle configured for alternatively engaging the tool shaft when the tool shaft is disengaged from the handle. In such embodiments, the T-handle defines a second cannula extending at least partially therethrough along the longitudinal axis. The T-handle may comprise a multilobular female connector disposed therein configured for fixed engagement with the plurality of teeth when the tool shaft is inserted into the second cannula.

### BRIEF DESCRIPTION OF THE DRAWINGS

FIG. 1 is a perspective view of a modular tool apparatus according to one exemplary embodiment.

FIGS. 2A and 2B are perspective views of the modular tool apparatus of FIG. 1 wherein the tool shaft (FIG. 2B) is removed from the handle (FIG. 2A).

FIG. 3 is a cross-section of the modular tool apparatus of FIG. 1

FIG. 4 is a partial perspective view of the modular tool apparatus of FIG. 1 with a portion of the handle removed to depict an elastic member disposed therein.

FIG. 5 is a partial perspective view of the modular tool apparatus of FIG. 1 with a further portion of the handle removed to depict components of an elastic member disposed therein.

FIG. 6 is a perspective view of a modular toot apparatus comprising a T-handle, according to one exemplary embodiment.

FIGS. 7A and 7B are distal end views of a tool shaft comprising polygonal male FIG. 7A) and female (FIG. 7B) drivers, according to one exemplary embodiment.

FIGS. 8A and 8B are distal end views of a tool shaft comprising muitilobular male (FIG. 8A) and female (FIG. 8B) drivers, according to one exemplary embodiment.

### DETAILED DESCRIPTION

The embodiments disclosed may be understood more readily by reference to the following detailed description taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value, When such orange is expressed, another embodiment includes from the one particular value and/or to the other particular value. Similarly, when values are expressed as approximations, by use of the antecedent “about,” it will be understood that the particular value forms another embodiment. It is also understood that all spatial and numerical references, such as, for example, horizontal, vertical, top, upper, lower, bottom, left and right, first and second, are for illustrative purposes only and can be varied within the scope of the disclosure. For example, the references “superior” and “inferior” are relative and used only in the context to the other, and are not necessarily “upper” and “lower”.

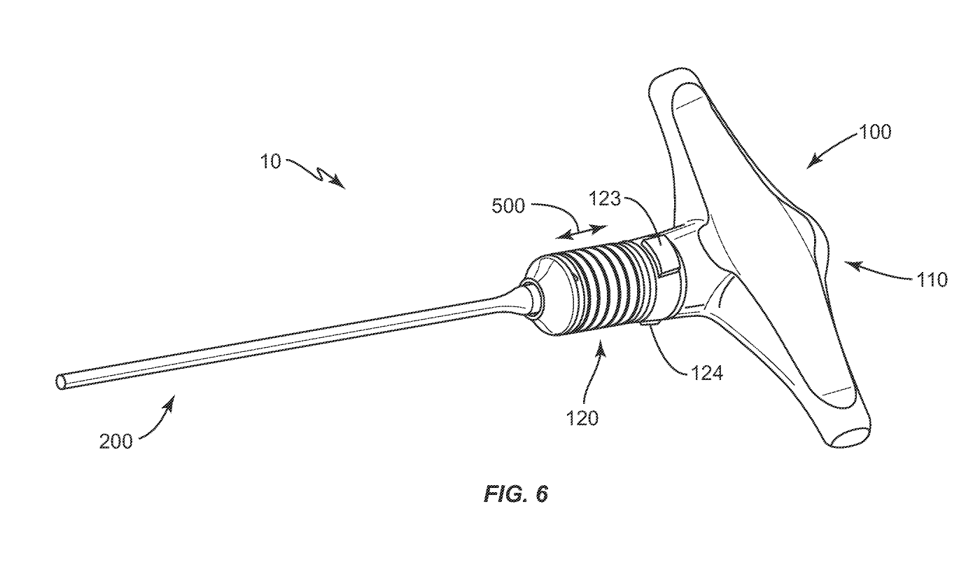

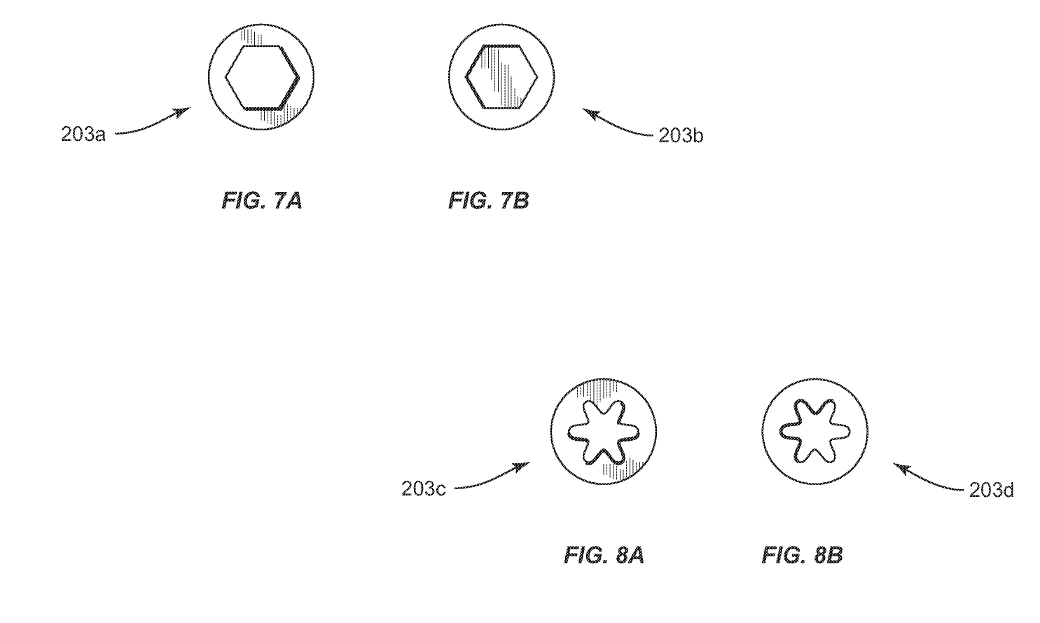

While the various embodiments of the modular tool apparatus 10 described herein are focused on use of the apparatus 10 as a driver tool (see FIGS. 7A, 7B, 8A and 8B, for example) it should be noted that the tool shaft 200 may comprise a variety of tool ends including, but not limited to: screw thread taps, drills, socket drivers, reamers, awls, punches, cam tools and combinations thereof Furthermore, the various embodiments of the modular tool apparatus 10 described herein may be used to facilitate a variety of surgical procedures including, but not limited to: driving bone screws (including, but not limited to pedicle screws); driving set screws (including, but not limited to set screws suitable for securing spinal rods into the proximal portion, or “tulip” of a fixed-angle and/or multi-axial pedicle screw); tapping screw threads into bone or other materials; drilling into bone or other materials; applying torque or rotational force to intervertebral implants having screws or other rotational components integrated therein for assembly and/or expansion; deploying anti-back out mechanisms disposed on bone plate assemblies (including, but not limited to cervical bone plates and occipital plates) and combinations thereof. For example, the various embodiments described herein may be provided in a kit format comprising a variety of tool shafts 200 having different distal ends (see the various drivers of FIGS. 7A, 7B, 8A and 8B, for example) and/or a variety of handles 100 (including, but not limited to a fixed T-handle 100 as shown in FIG. 6). Furthermore, the various components of the modular tool apparatus 10 may comprise any suitable material including, but not limited to: stainless steel, biocompatible polymers, titanium, titanium alloys, substantially-rigid engineering polymers, rubber, elastomeric polymers, spring steel, steel alloys, aluminum, aluminum alloys and combinations thereof.

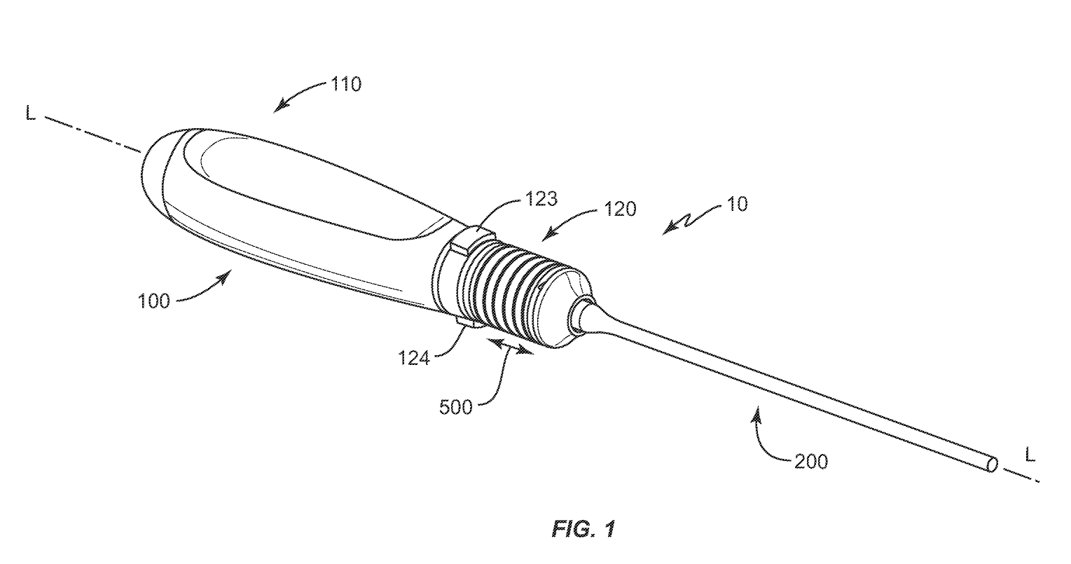

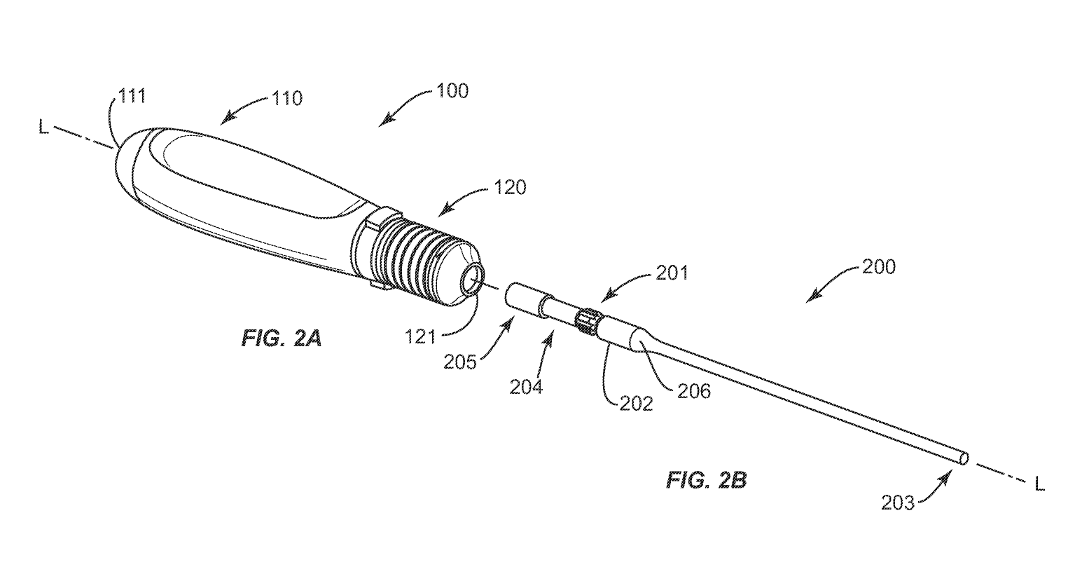

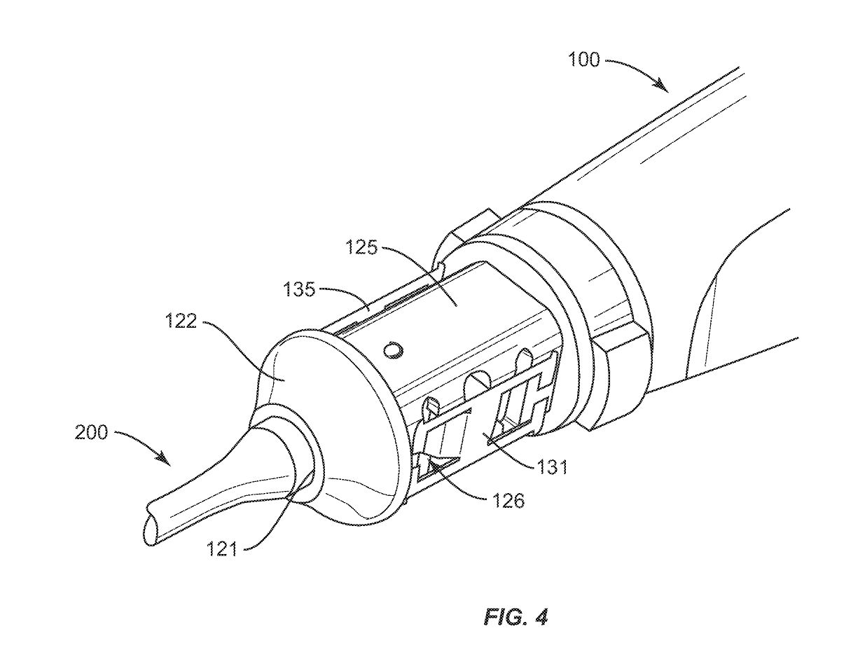

Referring to FIG. 1, an exemplary embodiment of a modular tool apparatus 11 is shown extending along a longitudinal axis L. The apparatus 10 comprises a handle 100 defining a cannula (See FIG. 2A, for example, depicting a distal opening 121 of the cannula defined in the handle 100) extending at least partially therethrough. along the longitudinal axis L. As shown generally in FIG. 4, the handle 100 my comprise one or more elastic members 131, 135 (such as a flat spring, for example) disposed therein. The modular tool apparatus 10 of FIG. 1 further comprises a tool shaft 200 configured to be releasably engaged with the handle 100 by insertion into the cannula 121. As shown in FIG. 1, the tool shaft 200 may comprise a plurality of teeth 201 configured for ratcheting engagement with the elastic member and/or members 131, 135 when the tool shaft 200 is inserted into a distal opening 121 forming a cannula, such that when the handle 100 is rotated about the longitudinal axis L in a first direction, the tool shaft 200 rotates with the handle 100 and such that when the handle 100 is rotated about the longitudinal axis L in a second direction, the tool shaft 200 rotates relative to the handle 100 (in a ratcheting engagement). Therefore, as shown, for example in FIGS. 2A and 2B, the ratcheting mechanism (comprising at least the plurality of teeth 201 disposed on the tool shaft and the various components of the elastic member(s) 131, 135 described herein) may be detachable and/or disassembled such that the various components of the modular tool apparatus 10 may be more easily subjected to cleaning procedures and/or sterilization procedures such that the modular tool apparatus 10 may be suitable for repeated re-use in surgical procedures.

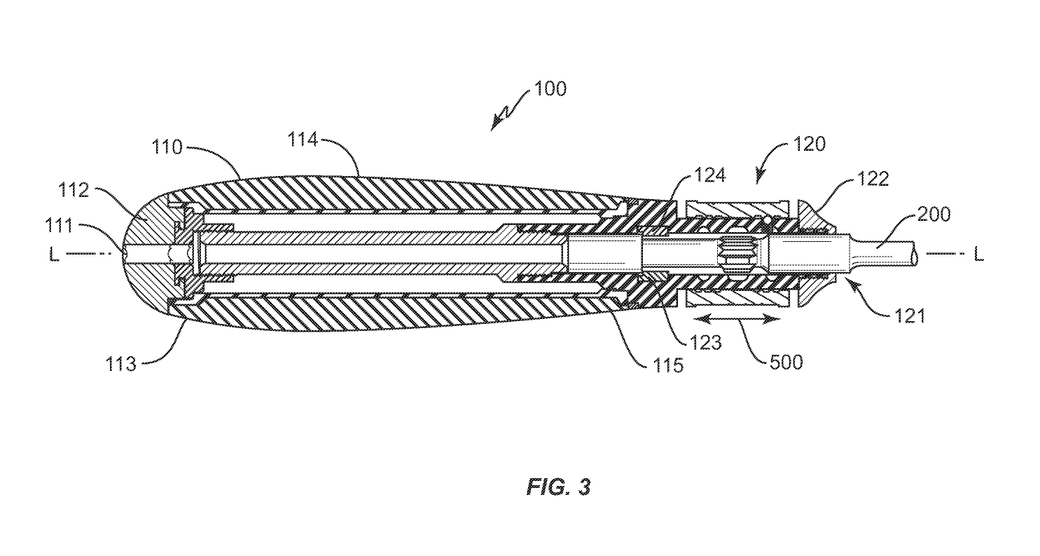

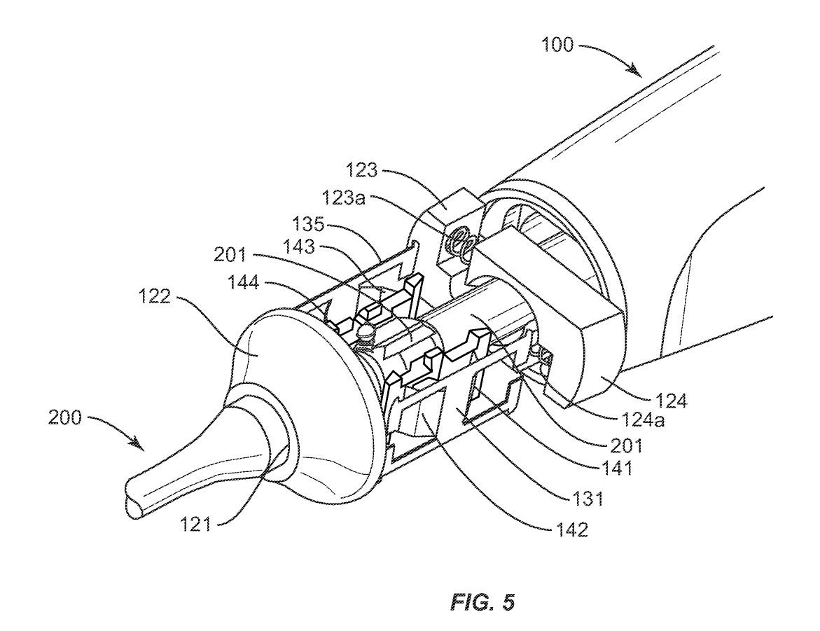

More particularly, as shown in FIGS. 2A and 2B, the handle 100 and the tool shaft 200 may be separable by depressing the retention members 123, 124 (see FIG. 5) and pulling the tool shaft 200 out of the distal opening 121 defining the cannula in the handle 100. In some such embodiments, the cannula may extend completely through the handle 100 along the longitudinal axis L from a proximal opening 111 (located on a proximal portion 110 of the handle 100) to the distal opening 121 (located on a distal portion 120 of the handle 100 or defined in a distal conical portion 122 (see FIG. 3)). As shown in FIG. 2A, the proximal portion 110 may comprise a polymeric and/or rubber grip suitable for manipulation by a user in the surgical setting. Furthermore, the distal portion 120 may comprise a metallic and/or metal alloy sleeve providing a rigid setting for the various components of the elastic members 131, 135 making up at least a portion of the ratchet mechanism.

Referring to FIG. 2B, the tool shaft 200 comprises a plurality of teeth 201 disposed and con⁻figured for ratcheting engagement with the elastic member and/or members 131, 135 when the tool shaft 200 is inserted into the distal opening 121, Furthermore, the tool shaft 200 includes a proximal end 205 and a distal end 203 which may comprise a fastener driving element that may include, but is not limited to: a multilobular male driver 203c (see FIG. 8A); a multilobular female driver 203d (see FIG. 8B); a fiat-head male driver (not shown); a Philips head male driver (not shown); a polygonal male driver 203a (see FIG. 7A); and a polygonal female driver 203b (see FIG. 7B. Furthermore the tool shaft 200 may also comprise a tapered portion 206 located distally to the plurality of teeth 201 disposed thereon. Referring again to FIG. 2B, the tool shaft 200 may also comprise a proximal segment comprising relatively wide portions 202, 205 and a narrow portion 204 (having a second diameter that is less than the first diameter defined by the wide portions 202, 205). The above-described proximal segment of the tool shaft 200 may be configured for optimal releasable engagement with the retention members 123, 124 (see FIGS. 3 and 5) of the handle 100 as described in further detail herein.

In some embodiments, as shown generally in FIG. 4, the elastic members 131, 135 may be housed near a distal end 122 of the handle 100 in a housing 125. FIG. 5 depicts the housing 125 removed from the handle 100 such that the various components of the ratchet assembly are more visible. For example, the elastic members 131, 135 housed within the handle may comprise one or more pawls 126 (see FIG. 4, showing the pawl 126 being urged inward by a leaf spring element extending from the elastic member 131) and springs 142, 141, 143, 144 configured to bias the pawl 126 towards the plurality of teeth 201 of the tool shaft 200 when the tool shaft 200 is inserted into the distal opening 121 of the handle 100. As shown in FIGS. 4 and 5, springs 142, 141, 143, 144 may comprise leaf springs or spring “arms” integrally formed with flat spring elastic members 131, 135 suitable for engagement between a housing 125 and the distal portion 120 of the handle such that the springs 142, 141, 143, 144 urge one or more pawls 126 toward the plurality of teeth 201 formed on the tool shaft 100. As will be appreciated by one of skill in the art, the pawl 126 may be formed to be “directional” such that the teeth 201 (and correspondingly, the tool shaft 200) may “ratchet” relative to the spring-loaded pawl 126 which may give way when the handle 100 is turned in a first direction. Furthermore, the pawl 126 may hold fast so as to fixedly engage the teeth 201 when the handle is turned in a second direction, opposite the first such that a turn of the handle 100 may induce a corresponding equal turn (and torque) on the tool shaft 200.

Referring to FIG. 5, in some embodiments of the modular tool apparatus 10, the handle 100 further comprises one or more retention members 123, 124 engaged therewith. The retention members 123, 124 may be configured for releasably engaging the tool shaft 200 with the handle 100 when the tool shaft 200 is inserted into the cannula formed at least in part by the distal opening 121 in the handle 100. In some embodiments, the retention members 123, 124 comprise tabs 123, 124 (or “buttons”) extending through the handle 100, wherein the tabs 123, 124 define a channel therein (the channel being at least partially co-axial with the cannula formed between the proximal opening 111 and the distal opening 121 defined in the handle, The tabs or buttons 123, 124 forming the retention members may also be opposed about the cannula so as to be engageable with the tool shaft 200. For example, the retention members 123, 124 may comprise biasing elements 123a, 124a configured for biasing the tabs 123, 124 in first position wherein the tabs 123, 124 engage at least a portion of the tab 123, 124 with the tool shaft 200 (such as a narrow portion 204 thereof) such that the tool shaft 200 is releasably engaged with the handle 100 within the cannula. Furthermore, according to some such embodiments, when the tabs 123 124 are pressed against the biasing elements 123a, 124a (urging the tabs 123, 124 toward one another, for example) to a second position, the tool shaft 200 is removable from the handle 100 along the longitudinal axis L (see FIGS. 2A and 2B, for example).

As noted herein, and referring to FIG. 2B, the tool shaft 200 may comprise a cylinder having a first diameter, and wherein the tool shaft comprises a distal end 203 and a proximal end 205, wherein the proximal end 205 has a second diameter larger than the first diameter such that the proximal end 205 is releasably retained within the channel defined in the retention member(s) 123, 124 when the tool shaft 200 is engaged with the handle 100, Referring again to FIG. 2B, the tool shaft 200 may comprise a tapered portion 206 distal to the plurality of teeth 201. In some such embodiments, the distal portion 203 may have the smaller first diameter and the tapered portion 206 may gradually increase in diameter to the second diameter of the proximal portion 205, Furthermore, a narrow portion 204 having the first diameter (or a third diameter larger than the first diameter, but smaller than the diameter of the proximal portion 205) may be interposed between the tapered portion 206 and the proximal portion 205 to provide an axial attachment region such that the tool shaft 200 may be releasably captured in the cannula (by the interaction of the retention members 123, 124, for example) of the handle 100. In such embodiments, when a user presses both of the retention members 123, 124 radially inward towards the longitudinal axis, the tool shaft 200 may be releasable from the handle 100 and moved in a direction 500 along the longitudinal axis (see FIGS. 1, 2A and 2B, for example).

Referring now to FIG. 3, showing a cross-sectional view of the handle 100 according to one embodiment, the handle 100 may comprise an inner rigid frame (made of a metal alloy or other suitable engineering material as described further herein) configured for retaining the elastic members 131, 135 (see FIG. 4). The frame may comprise an inner portion of the handle 100 that may include the housing 125 depicted generally in FIG. 4 and other substantially rigid components that may be threadably engaged with the handle 100 (such as a proximal end cap 112, and a distal conical member 122). As shown generally in FIG. 3, an inner frame of the handle may define the cannula extending completely through the handle 100 along the longitudinal axis L from the proximal opening 111 to the distal opening 121. As shown in FIG. 3, the handle 100 may also comprise an outer handle 114, 115 surrounding the inner frame and suitable for gripping by a user. The outer handle 114, 115 may comprise a suitable polymeric and/or rubberized material as described more fully herein for gripping by a user in a surgical setting. The distal portion 120 of the handle 100 may also comprise a removable and/or separable metallic sleeve suitable for retaining and/or protecting the elastic members 131, 135 within the handle 100.

In some embodiments as shown in FIG. 6, the modular tool apparatus 10 may further comprise a T-handle 100 configured for alternatively engaging the tool shaft 200 when the tool shaft is disengaged from the straight, ratcheting handle 100 shown in FIGS. 1-5. In some such embodiments, the T-handle may define a second cannula extending at least partially therethrough along the longitudinal axis. The T-handle 100 may also comprise a multilobular female connector (similar to the arrangement shown in FIG. 8B) disposed therein configured for fixed engagement with the plurality of teeth 201 when the tool shaft 200 is inserted into the second cannula defined in the T-handle. In some alternative, embodiments, the T-handle 100 may comprise the various ratcheting components described herein with reference to FIGS. 1-5. For example, the distal portion 120 of the T-handle embodiment may also house one or more elastic members 131, 135 configured for ratcheting engagement with the plurality of teeth 201 disposed on the tool shaft 200. Furthermore, as shown in FIG. 6, the T-handle embodiments may further comprise retention members 123, 124 engaged therewith, wherein the retention members 123, 124 are configured for releasably engaging the tool shaft 200 with the T-handle when the tool shaft 200 is inserted into the cannula thereof.

Also, it should be understood that each of the above-described embodiments may be combined in whole or in part with one or more of the other above-described embodiments. It is further understood that each of the above-described embodiments may be combined in whole or in part with other components, devices, systems, methods and/or surgical techniques known to those skilled in the art to provide spinal stabilization or to perform other medical procedures.

Although exemplary embodiments have been described in detail above, those skilled in the art will readily appreciate that many other modifications are possible in the exemplary embodiments without materially departing from the novel teachings and advantages described herein. Accordingly, all such modifications are intended to be included within the scope defined by the following claims.

What is claimed is:

1. A modular tool apparatus extending along a longitudinal axis, the apparatus comprising:
a handle defining a cannula extending at least partially therethrough along the longitudinal axis, the handle comprising an elastic member disposed therein;
a tool shaft configured to be releasably engaged with the handle by insertion into the cannula, the tool shaft comprising a plurality of teeth configured for ratcheting engagement with the elastic member when the tool shaft is inserted into the cannula such that when the handle is rotated about the longitudinal axis in a first direction, the tool shaft rotates with the handle and such that when the handle is rotated about the longitudinal axis in a second direction, the tool shaft rotates relative to the handle.

2. The apparatus of claim 1 wherein elastic member comprises a pawl and a spring configured to bias the pawl towards the plurality of teeth of the tool shaft when the tool shaft is inserted into the cannula.

3. The apparatus of claim 1, wherein the handle further comprises a retention member engaged therewith, the retention member configured for releasably engaging the tool shaft with the handle when the tool shaft is inserted into the cannula.

4. The apparatus of claim 3, wherein the retention member comprises:
a tab extending through the handle, the tab defining a channel therein, the channel being at least partially co-axial with the cannula; and
a biasing element configured for biasing the tab in a first position wherein the tab engages at least a portion of the tab with the tool shaft such that the tool shaft is releasably engaged with the handle within the cannula;
wherein when the tab is pressed against the biasing element to a second position, the tool shaft is removable from the handle along the longitudinal axis.

5. The apparatus of claim 3, wherein the tool shaft is a cylinder having a first diameter, and wherein the tool shaft comprises a distal end and a proximal end, wherein the proximal end has a second diameter larger than the first diameter such that the proximal end is releasably retained within the channel of the retention member when the tool shaft is engaged with the handle.

6. The apparatus of claim 1, wherein the tool shaft comprises a tapered portion distal to the plurality of teeth.

7. The apparatus of claim 1, wherein the tool shaft comprises a distal end and a proximal end, the distal end comprising a fastener driving element.

8. The apparatus of claim 7, wherein the fastener driving element is selected from the group consisting of:
a multilobular male driver;
a multilobular female driver;
a flathead male driver;
a Philips head male driver a polygonal male driver; and
a polygonal female driver.

9. The apparatus of claim 1, wherein the handle comprises an inner rigid frame configured for retaining the elastic member and an outer handle.

10. The apparatus of claim 1, further comprising a T-handle configured for alternatively engaging the tool shaft when the tool shaft is disengaged from the handle, the T-handle defining a second cannula extending at least partially therethrough along the longitudinal axis, the T-handle comprising a multilobular female connector disposed therein configured for fixed engagement with the plurality of teeth when the tool shaft is inserted into the second cannula.

11. A modular tool apparatus extending along a longitudinal axis, the apparatus comprising:
a handle having a proximal end and a distal end, the handle defining a cannula extending completely therethrough from the proximal end to the distal end, along the longitudinal axis, the handle comprising an elastic member disposed therein;
a tool shaft configured to be releasably engaged with the handle by insertion into the cannula from the distal end of the handle, the tool shaft comprising a plurality of teeth configured for ratcheting engagement with the elastic member when the tool shaft is inserted into the cannula such that when the handle is rotated about the longitudinal axis in a first direction, the tool shaft rotates with the handle and such that when the handle is rotated about the longitudinal axis in a second direction, the tool shaft rotates relative to the handle.

12. The apparatus of claim 11 wherein elastic member comprises a pawl and a spring configured to bias the pawl towards the plurality of teeth of the tool shaft when the tool shaft is inserted into the cannula.

13. The apparatus of claim 11, wherein the handle further comprises a retention member engaged therewith, the retention member configured for releasably engaging the toot shaft with the handle when the tool shaft is inserted into the cannula.

14. The apparatus of claim 13, wherein the retention member comprises:
a tab extending through the handle, the tab defining a channel therein, the channel being at least partially co-axial with the cannula; and
a biasing element configured for biasing the tab in a first position wherein the tab engages at least a portion of the tab with the tool shaft such that the tool shaft is releasably engaged with the handle within the cannula;
wherein when the tab is pressed against the biasing element to a second position, the tool shaft is removable from the handle along the longitudinal axis.

15. The apparatus of claim 14, wherein the tool shaft is a cylinder having a first diameter, and wherein the tool shaft comprises a distal end and a proximal end, wherein the proximal end has a second diameter larger than the first diameter such that the proximal end is releasably retained within the channel of the retention member when the tool shaft is engaged with the handle.

16. The apparatus of claim 11, wherein the tool shaft comprises a tapered portion distal to the plurality of teeth.

17. The apparatus of claim 11, wherein the tool shaft comprises a distal end and a proximal end, the distal end comprising a fastener driving element.

18. The apparatus of claim 17, wherein the fastener driving element is selected from the group consisting of:
a multilobular male driver;
a multilobular female driver;
a fiat-head male driver;
a Philips head male driver
a polygonal male driver; and
a polygonal female driver.

19. The apparatus of claim 11, wherein the handle comprises an inner rigid frame configured for retaining the elastic member and an outer handle.

20. The apparatus of claim 11, further comprising a T-handle configured for alternatively engaging the tool shaft when the tool shaft is disengaged from the handle, the T-handle defining a second cannula extending at least partially therethrough along the longitudinal axis, the T-handle comprising a multilobular female connector disposed therein configured for fixed engagement with the plurality of teeth when the tool shaft is inserted into the second cannula.
